(19) United States
(12) Patent Application Publication
(10) Pub. No.: US 2013/0245664 A1
(43) Pub. Date: Sep. 19, 2013

(54) CERVICAL DILATOR

(71) Applicant: Karen Louise Niver, Port Huron, MI (US)

(72) Inventor: Karen Louise Niver, Port Huron, MI (US)

(73) Assignee: Raggio & Dinnin, P.C., Auburn Hills, MI (US)

(21) Appl. No.: 13/798,343

(22) Filed: Mar. 13, 2013

Related U.S. Application Data
(60) Provisional application No. 61/610,686, filed on Mar. 14, 2012.

(51) Int. Cl. A61M 29/02

(52) U.S. Cl. CPC A61M 29/02
USPC 606/193

(57) ABSTRACT

A cervical dilator for use in dilating a cervix comprises a light weight flexible tube member. The cervical dilator also comprises a syringe connected to a first end of the flexible tube member for introducing a liquid or air solution into the tube member. The cervical dilator also includes a balloon arranged over an end of the tube that is inserted into the cervix of the patient in an uninflated state. The cervical dilator also comprises at least one orifice arranged through or near an end of the flexible tube member and within the interior cavity of the balloon, such that the air or liquid inflates the balloon to a predetermined diameter.

### BACKGROUND OF THE INVENTION

1. Field of the Invention

The present invention generally relates to an instrument used for cervical dilation and more particularly relates to an improved cervical dilator that streamlines the surgical procedure of dilating a uterine cervix thus benefiting the patient, physician and surgical team in the operating environment.

2. Description of Related Art

Prior art cervical dilators, such as Hanks dilators, have been used by obstetrician/gynecologists (OB/GYNs) for many years. Generally, a Hanks dilator set is comprised of a set of six metal rods, each having a specific graduated diameter on each end thereof. The use of Hanks dilators to dilate a uterine cervix requires twelve insertions and twelve points of potential damage and infection to the patient during the process. Generally, when a women presents to a OB/GYN with abnormal uterine bleeding, her doctor is required to investigate the source of such uterine bleeding, in order to accomplish this investigation, many of today's OB/GYN's use the prior art process of handling six separate instruments to make-twelve separate, step wise insertions through the cervix to gain access to the uterine cavity. These prior art instruments are used to increasingly spread open or dilate the uterine cervix in order to allow the OB/GYN to see into and investigate the source of the bleeding within the uterine cavity. The prior art Hanks dilators are also used to begin the process of a dilation and curettage (D&C) that is performed for a woman who has suffered a miscarriage.

However, the use of these dilator sets presents several technological constraints to the physician using such instruments. The Hanks dilators are rigid instruments that inherently risk damage to the cervix, uterus, bowel bladder and major blood vessels by puncture or perforation. The use of the Hanks dilators also creates an excessive number of passes or insertions, in most cases twelve, to fully dilate the cervix, thus increasing the risk associated with infection or the creation of unintended false passages within the uterine fundus. Furthermore, the use of the Hanks dilators and the current one size fits all approach does not customize to the individual patient's cervical length. The use of the prior art dilators creates a multi step process that allows for potential contamination of the sterile dilators and other equipment used during the procedures on the uterus. Additionally, a twelve step process may introduce potential human error via incorrect packaging of instruments, possible out of sequence handling or insertion by the doctor or by assistants or nurses assisting during the procedure. There are many potential sites of perforation within the female pelvic anatomy. Some of these include the uterine fundus, the uterine cervix, the bladder, the rectum, and major blood vessels of the female pelvic anatomy. With each insertion of the prior art dilators the risk of such potential perforations increases, thus increasing the risk to the patient having the procedure performed thereon.

Another risk associated with using prior art metal dilators is that human error may occur in the packaging of and the sterilization of the instruments offered to the physician during the procedure. With each of the dilators increasing by a small percentage in diameter, the possibility of receiving a dilator in an improper order occurs. Rigid metal dilators have been used throughout history for performing cervical dilations, and few other technologies have been able to gain a foothold into such cervical dilation procedures by OB/GYN's.

Therefore, there is a need in the art for an improved cervical dilator that may be custom, fitted using flexible materials for each patient's cervix length. There also is a need in the art for an improved cervical dilator that uses warm saline infusion and balloon technology to dilate the cervix through the insertion of one instrument, thus creating only one insertion into the woman's cervical cavity. Furthermore, there is a need in the art for a cervical dilator that has graduated and ratcheted inflation by one instrument to create the same graduated dilation that was done by six instruments via twelve insertions of the prior art. There also is a need in the art for a cervical dilator that has the ability to measure the uterine length, thus avoiding a thirteenth insertion into the dilated uterus. Furthermore, there is a need in the art for a disposable cervical dilator that may eliminate confusion and human error along with sterilization concerns of the dilation mechanism. Hence, there also is a need in the art to develop a safer, more cost-effective cervical dilator instrument while also streamlining the surgical procedure of dilating the uterine cervix, thus benefiting the patient, physician and the entire surgical team during such procedures.

### SUMMARY OF THE INVENTION

One object of the present invention may be to provide an improved cervical dilator.

Another object of the present invention may be to provide a cervical dilator that is custom fitted for each individual patient using flexible materials.

Still another object of the present invention may be to provide a cervical dilator that uses balloon technology along with a warm saline infusion to dilate the cervix.

Still another object of the present invention may be to provide an improved cervical dilator that has graduated and ratcheted inflation via one instrument inserted in the cervix as opposed to the prior art six instruments that were inserted twelve times to dilate the cervix to allow for proper viewing and surgery therein by the physician.

Still another object of the present invention may be to provide a cervical dilator that has the ability to measure the uterine length without inserting another instrument into the dilated uterus.

Yet another object of the present invention may be to provide a disposable cervix dilator that eliminates confusion and human error during cervical dilation.

Still another object of the present invention may be to provide a cervical dilator that decreases the risk of cervical damage and future cervical incompetence along with decreasing the risk of uterine perforation.

Still another object of the present invention may be to provide a cervical dilator that decreases the risk of damage to surrounding organs while also decreasing the risk of false track formation during the procedure.

Yet another object of the present invention may be to provide a cervical dilator that decreases the risk of vascular damage, bleeding and infection by making the cervical dilation a quicker procedure than that of the prior art.

Still another object of the present invention may be to provide a cervical dilator that creates a less complicated and time consuming procedure for the physician.

Still another object of the present invention may be to provide a cervical dilator that generally eliminates the risk of improperly packaged instruments and of improper sequencing and insertion of instruments into the cervix.

Still another object of the present invention may be to provide a cervical dilator that decreases the risk of contamination of the patient by the cervical dilation instruments.

According to the present invention, the foregoing and other objects and advantages are obtained by a novel design for a cervical dilator. The cervical dilator generally comprises a tube having a light weight hand grip arranged cm one end thereof. The cervical dilator also includes a syringe that has ratcheting arranged on an inner surface thereof, connected to the tube of the cervical dilator for introducing a warm saline solution into the tubular member. The cervical dilator also includes on one end of the tube a balloon. The balloon is arranged over the end of the tube that is inserted into the cervix of the patient. The balloon is inserted into die cervix in an uninflated state. Upon insertion into the cervix, the balloon may be inflated via the warm saline solution or any other type of inflation mechanism, such as air, or other liquids or gasses, thus dilating the cervix to the appropriate width as deemed necessary by the physician. The cervical dilator also includes a ring or collar arranged over the surface of the tube and the uninflated balloon before insertion into the cervix of the patient. The ring or collar is movable with relation to the end of the tube, thus creating an adjustable dilator depending on the measurement of the cervical canal by the physician during the procedure. It should be noted that all of the components of the cervical dilator are generally made of a sterilized plastic material that is hypoallergenic, thus decreasing the risk of reactions to the instrument by the patient. It should be noted that the tubular member of the cervical dilator is flexible thus allowing for a variety of different shaped cervices and uteri.

One advantage of the present invention may be that it provides for an improved cervical dilator.

A further advantage of the present invention may be that it provides for an improved cervical dilator that is made of sterilized plastic or metal components that creates a one time use instrument, thus ensuring sterilization of the cervical dilator.

Still a further advantage of the present invention may be that it provides for a cervical dilator that has custom fitting via the use of flexible materials.

Still a further advantage of the present invention may be that it provides for a cervical dilator that uses balloon technology and a warm saline infusion to dilate the cervix of the patient.

Still another advantage of the present invention may be that it provides for a cervical dilator that has graduated and ratcheted inflation capabilities, thus allowing only one instrument as opposed to the prior art six to dilate the cervix and uterus to the proper diameter. This also creates only one insertion by the physician as opposed to twelve via the prior art dilation methodology.

Still another advantage of the present invention may be that it provides for a cervical dilator that has the ability to measure the uterine length, thus avoiding a thirteenth insertion such as that done in prior art to determine such length.

Yet another advantage of the present invention may be that it provides for a cervical dilator that has a disposable feature, thus eliminating confusion and human error by the operators.

Still another advantage of the present invention may be that it provides for a cervical dilator that decreases the risk of cervical damage and future cervical incompetence while also decreasing the risk of uterine perforation

Still another advantage of the present invention may be that it provides for a cervical dilator that decreases the risk of damage to surrounding organs and false track formation.

Still another advantage of the present invention may be that it provides for a cervical dilator that decreases the risk of vascular damage, bleeding and infection by reducing the amount of time for the procedure, thus making a quicker procedure.

Still another advantage of the present Invention may he that it provides far a cervical dilator that creates a less complicated procedure and time consuming procedure for the physician, thus reducing the risk of any damage to the patient.

Still another advantage of the present invention may be that it provides for a cervical dilator that eliminates the risk of improperly packaged instruments and improper sequencing of instruments being placed into the cervix of the patient.

Still another advantage of the present invention may be that it provides for a cervical dilator that decreases the risk of contamination to the patient.

Yet a further advantage of the present invention may be that it provides for an improved cervical dilator that is a more cost effective instrument which streamlines the surgical procedure of dilating the uterine cervix, thus benefiting the patient, physician, and the entire surgical team.

Other objects, features and advantages of the present invention may become apparent, from the subsequent description and appended claims, taken in conjunction with the accompanying drawings/

### BRIEF DESCRIPTION OF THE DRAWINGS

FIG. 1 shows a cross sectional view of the female pelvic anatomy with a prior art cervical dilator inserted into the cervix.

FIG. 2 shows a side view of a set of prior art Hank dilators.

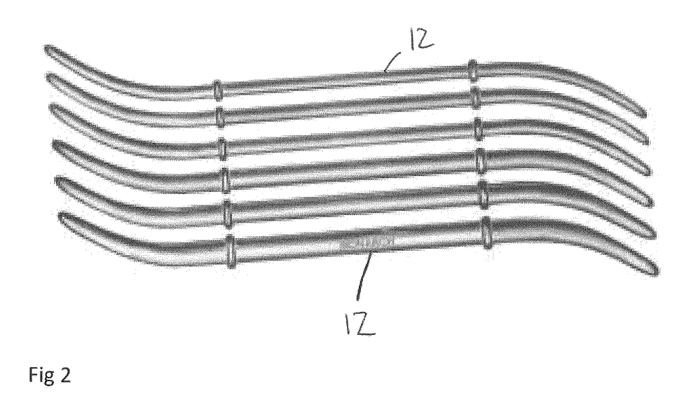

FIG. 3 shows a side view of an embodiment of a cervical dilator according to the present invention.

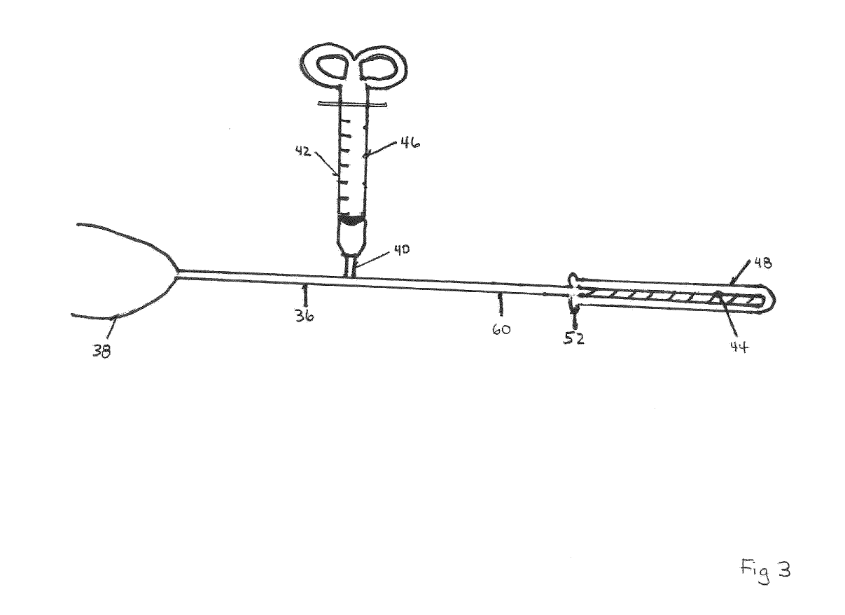

FIG. 4 shows a cross sectional view of an embodiment of a cervical dilator according to the present invention.

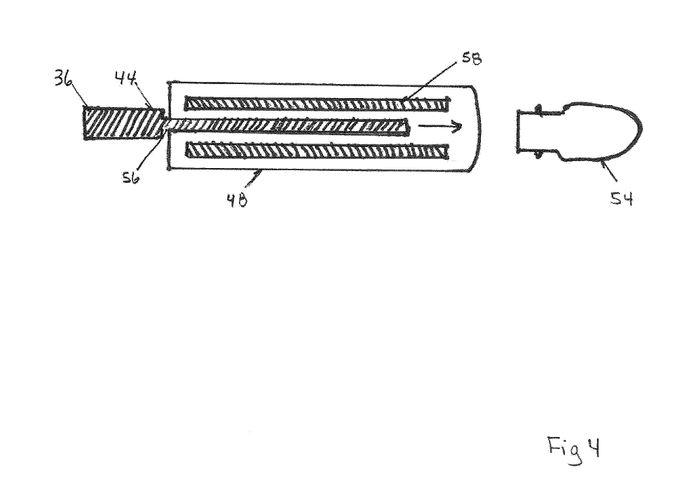

FIG. 5 shows a close up of a fully assembled lip of art embodiment of a cervical dilator

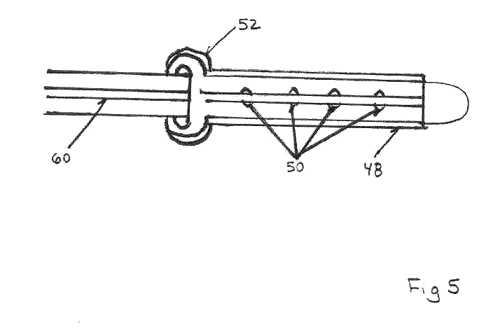

according to the present invention.

FIG. 6 shows a plan view of an alternate embodiment of a cervical dilator according to the present invention.

### BRIEF DESCRIPTION OF THE EMBODIMENT

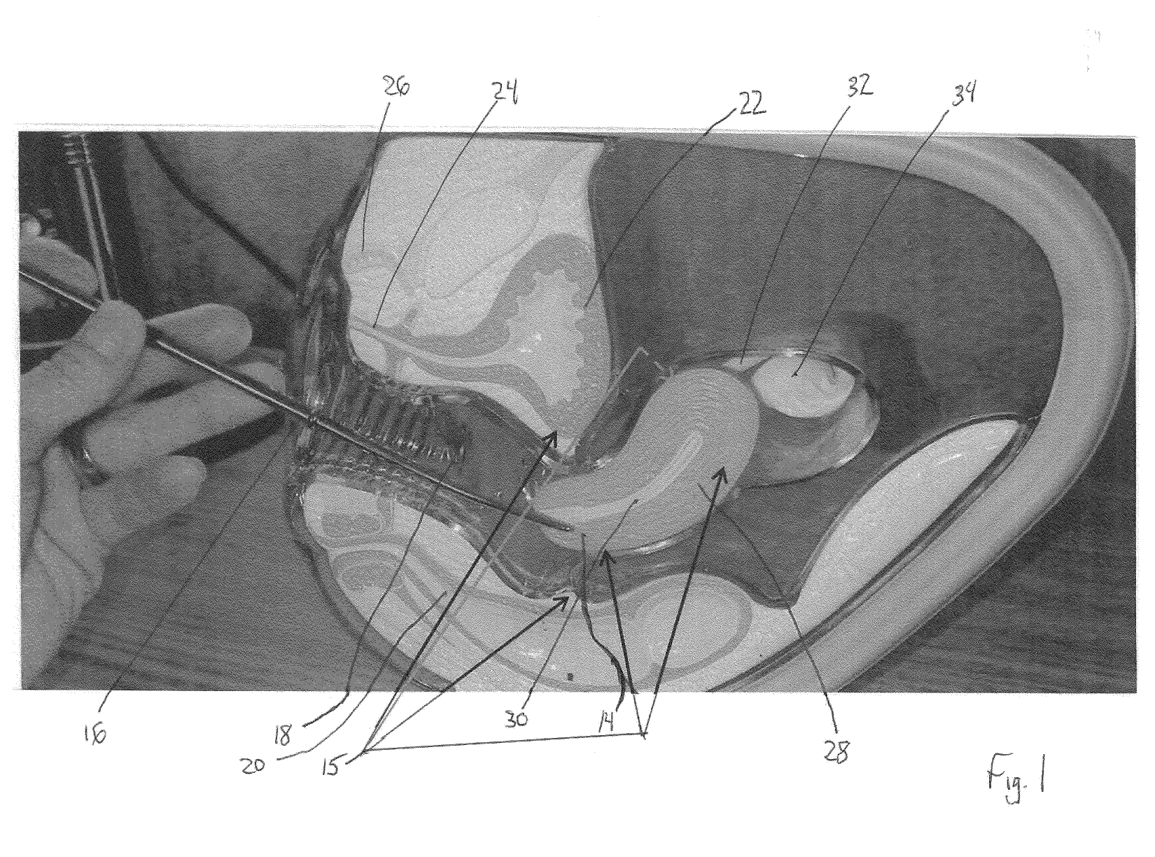

Referring to the drawings, there is shown a cervical dilator 10 according to an embodiment of the present invention. The drawings also show an associated methodology for use in dilating a cervix of a patient with the cervical dilator 10 of the present invention. FIG. 1 shows a cross section of a female pelvic anatomy having a prior art Hanks dilator 12 inserted into the cervix 14. Generally, the female pelvic anatomy includes the labia 16, which flanks the outside portion of a female vagina 18. The female vagina 18 is located directly adjacent to the labia 16 in the female pelvic anatomy. A female's rectum 20 and bladder 22 are arranged directly adjacent to the vagina 18 of the female. The bladder 22, urethra 24 and clitoris 26 are all arranged above the vagina 18 in a female, while the rectum 20 is arranged below the vagina 18 in the female pelvic anatomy. Arranged a variable distance within the vagina 18 is the cervix 14 and uterus 28. The uterus' front portion is commonly called the cervix 14, while the rear portion of the uterus is commonly called the uterine fundus 28. Arranged along a mid point thereof is the endometrial canal 30 into which an egg that has been fertilized by the sperm of a male is implanted during a pregnancy of the female. The endometrial canal 30 has a variable length and generally has a slightly curved shaped when viewed from a side as shown in FIG. 1. Directly connected to the uterus 28 and extending from each side thereof, are fallopian tubes 32 that are directly approximated to an ovary 34. The ovary 34 holds the eggs of the female, which are passed through the fallopian tubes 32 to the uterus 28 on a monthly cycle.

Generally, cervical dilation is performed when a female presents to her physician with abnormal uterine bleeding. The physician generally is required to investigate the source of such bleeding and in order to accomplish this most physicians currently use Hanks dilators 12 to complete the dilation of the cervix 14 to investigate the cause of the uterine bleeding. This process of cervical dilation with the use of the Hanks dilators 12 requires the physician to handle six separate instruments having different size diameters on each end thereof in a predetermined twelve separate, step wise insertion of the end diameters in an increasingly larger pattern through the cervix 14 in order to gain access to the uterine cavity 30 in order to allow for determination of the abnormal uterine bleeding. The abnormal uterine bleeding may have many different etiologies including miscarriage, and a dilation and curettage (D&C) or a uterine scraping must he performed by the physician. In such cases the physician may have to perform cervical dilation in order to accomplish the procedures thereon. The prior art methodology of performing the cervical dilation hence requires twelve insertions and twelve points of potential damage and infection to the patient in order to investigate the cause of abnormal bleeding from the uterus 28. Thus, the current procedure using the Hanks dilators 12, all six of them, requires twelve separate insertions and points of potential damage 15 to the cervix, uterus, bowel and bladder, major blood vessels increasing the risk of bleeding. Furthermore, the excessive number of passages, in this case twelve, increases the risk associated with infection and the creation of unintended false passages within the uterine fundus 28 or uterine cervix 14. Thus, this prior art one size fits all approach does not customize itself to the individual patients cervical length and the multi step process also allows for potential contamination of the sterile equipment during the procedure and misuse of the equipment by the physician by using the Hanks dilators 12 in the wrong step manner. The twelve step process currently used to perform a cervical dilation introduces potential human error via the incorrect packaging of the instruments, and possible out of sequence insertions by the physician.

The improved cervical dilator 10 of the present invention overcomes these drawbacks of the prior art methodology and instruments in performing a cervical dilation. As shown in the figures the cervical dilator 10 of the present invention includes a tube member 36 generally made of a plastic material that is flexible, sterile and hypoallergenic. It should be noted that the tube 36 may be made of any known material that is capable of being sterilized and hypoallergenic, thus not causing any adverse reactions to a patient having cervical dilation performed thereon. It is also contemplated to use any other type of metal, ceramic, composite or natural material to form the tube 36 of the cervical dilator 10 according to the present invention. Arranged on one end of the tube 36 may be a hand grip 38 that may allow for the physician to insert the cervical dilator 10 in a comfortable and easy manner into the cervix 14 of the uterus 28 of the patient. In one contemplated embodiment the hand grip 38 may be a textured light weight hand grip 38 made of a plastic or other material, however any other type of material may be used for the hand grip 38. The hand grip 38 may include texture as described above, or non texturing depending on the physician and the use therein. The tube 36 may have any known length and diameter. A generally cylindrical shaped port 40 extends from a surface of the tube member 36 of the cervical dilator 10. This port 40 may have, a predetermined diameter and length and may allow for the insertion of a syringe 42 into an end of the port 40. The inner diameter of the port 40 may generally match and be the same as the outer diameter of the syringe 42 nozzle that is arranged therein in order to create an airtight seal between the syringe 42 and the interior of the tube 36 of the cervical dilator 10. It is also contemplated to maybe have a nipple or valve member arranged within the port 40 that may or may not be spring activated that may allow for insertion of a gas or liquid into the tube 36 of the cervical dilator 10 only upon compression of the valve within the port 40 of the cervical dilator 10. Thus, upon removing the syringe 42 from the port 40 of the cervical dilator 10, a spring may expand and thus close the port 40, via the valve, to outside air creating an airtight cervical dilator 10. It should be noted that the port 40 is generally made of the same material as the tube member 36 of the cervical dilator 10. The end of the tube member opposite of the hand grip 38 may generally have a rounded end 44 with a predetermined diameter and orifice in the end thereof. In one contemplated embodiment the end 44 of the tube member 36 of the cervical dilator 10 may have a diameter of approximately two millimeters. However, it should be noted that the diameter of the end 44 of the cervical dilator 10 may be anywhere from one millimeter to ten millimeters, depending on the procedure with which the physician will use the dilator 10. It should be noted the end 44 of the tube 36 may not have an orifice therethrough. It should be noted that the material the tube member 36 is made of is generally a flexible material that may allow for the cervical dilator 10 to be inserted into any shaped or sized endometrial canals 30.

The cervical dilator 10 of the present invention also includes a syringe 42 which is generally arranged in the port 40 of the tube member 36 of the cervical dilator 10. In one contemplated embodiment the syringe 42 may have ratcheting notches 46 arranged on an inner surface thereof. These notches 46 may ratchet the syringe 42 in predetermined intervals, thus controlling the flow of liquid or gas being pumped through the syringe 42 into the tube 36 of the cervical dilator 10. In one contemplated embodiment the syringe 42 may contain a warm saline solution, which may be injected into the flexible tubular member 36 of the cervical dilator 10. It should be noted that any other liquid or gas may be used to dilate the cervix 14 of the patient with the present application. It is contemplated to use a warm saline solution to make the comfort of the patient as high as possible. It should further be noted that any type of gas, such as air, may also be used to inflate the balloon 48 which is arranged at the end 44 of the flexible tube member 36 of the cervical dilator 10. It should be noted that any type syringe 42 along with any sire syringe 42 may be used, ft is contemplated to use a plastic syringe, but any known metal, composite, or natural material syringe may also be used.

The cervical dilator 10 also includes a balloon 48 arranged over a predetermined length of the end of the flexible tube member 36 of the cervical dilator 10. It should be noted that generally the flexible tube member 36 has a bore or cavity 60 arranged along the entire inner portion or center line thereof, hence the warm saline solution which is introduced into the flexible tube member 36 may be able to be released from the tube member 36 into the balloon 48 in a predetermined manner depending on the ratcheting system of the syringe 42. The balloon 48 is generally arranged uninflated over the end 44 of the flexible tabular member 36. This uninflated balloon 48 may be inserted into the cervix 14 and the endometrial canal 30 a predetermined distance as measured by the physician via markings arranged on the outer surface of the flexible tubular member 36. This may allow for the physician to know the length of the cervical canal and endometrial canal 30 and how for the balloon 48 should be inserted into the endometrial canal 30 in order to allow for full dilation of the cervix 14. The balloon 48 may be secured on both ends thereof to allow for an air tight balloon 48 to be inflated via either injected air or liquid by the syringe 42. In one contemplated embodiment the balloon 48 is made of any known surgical plastic that is hypoallergenic and sterile to a patient's body. Such surgical balloons are well known in the art and have been used in surgical ports used in laparoscopic surgery and the like. It should be noted that in one contemplated embodiment a predetermined length of the end of the flexible tubular member 36 may have a plurality of orifices 50 arranged therein to allow for passage of the saline solution into the balloon member 48 in a uniform manner. However, it is also contemplated to have the saline solution or air exit from the just the very end of the flexible tubular member 36 to inflate the balloon 48 in a predetermined manner. The cervical dilator 10 also may include a ring or collar 52 arranged over the end of the flexible tubular member 36 a predetermined distance from the end 44 of the tubular member 36. This ring or collar 52 generally may be made of a plastic material. However, it should be noted that any other rubber, ceramic, composite, metal or natural material may also be used for both the balloon 48 and the ring 52. The ring 52 may be placed over one end of the uninflated balloon 48 to ensure correct placement of the balloon 48 during the dilation procedure of the cervix 14. The ring 52 generally may have an inner diameter that is equal to or slightly less than the outer diameter of the end of the flexible tubular member 36 of the cervical dilator 10. This may ensure an air tight seal for one end of the uninflated balloon 48 upon insertion into the cervical cavity 14 by the physician. Generally during the procedure, the ring 52 may be placed up against the outer portion of the cervix 14 of the uterus 28 before inflation of the balloon sleeve 48 via the saline solution. It should be noted that the inflated approximate range for the balloon diameter, with either air or saline solution, generally is five millimeters to twenty millimeters allowing for the physician to be able to see directly into the endometrial canal 30 and the uterus 28 of the patient. The balloon 48, as discussed above, may be inflated by the physician by pressing the plunger of the syringe 42 into the barrel of the syringe 42 via the ratcheting and/or markings 46 of the syringe 42 at a predetermined manner to inflate the balloon 48 to one of its adjustable widths, i.e., between five millimeters and twenty millimeters depending upon the patient who is having the cervical dilation. The other or outer end of the balloon 48 may be sealed by either a tip or sleeve 54 which is arranged within an end of the flexible tubular member 36 or by the end of the balloon 48 itself. This may allow for the physician via one insertion of the flexible tube 36 of the cervical dilator 10 of the present invention to completely dilate and measure the cervix 14 and uterus 28 with one instrument and one insertion instead of the twelve insertions and thirteenth insertion for measurement as done in the prior art. The physician would then remove the air or liquid by pulling the plunger of the syringe 42 up with relation to the barrel thus retracting the liquid or air back into the syringe 42 and deflating the balloon 48 enough to allow for removal of the cervical dilator 10 from the cervix 14 of the patient and allowing for the physician to insert an instrument within the uterus 28 for diagnostic or therapeutic purposes as per usual procedure.

It is also contemplated to have an embodiment of the end of the cervical dilator 10 and specifically the flexible tubular member end 44 that may include a reduced diameter extension 56 extending from the end 44 of the flexible tubular member 36. A sleeve 58 may then be arranged over the extension 56 with the balloon 48 arranged over both ends of the sleeve 58 thus allowing for the collapsed and uninflated balloon 48 to be secured with an air light seal between the end 44 of the flexible tubular member 36 of the cervical dilator 10 and an end of the sleeve 58. Any known mechanical securing techniques may be used to secure the sleeve 58 over the extension 56 extending from the end 44 of the flexible tubular member 36. A lip and notch system arranged on either of the extension 56 or the sleeve 58 may create such a connection that may seal one end of the balloon 48 with relation to the flexible tubular member 56.

On the opposite end of the sleeve 58 may be inserted a tip 54 that may also create an air tight seal and hold the uninflated or collapsed balloon 48 with relation to the end of the flexible tubular member 36. Any known mechanical connection may secure the tip 54 to the sleeve 58 on one end thereof. It should also be noted that the extension 56 extending from the end of the tubular member 36 may be integrated or formed or injection molded as a piece of the tube 36 or may be added as a separate piece after creation of the lobular member 36 of the cervical dilator 10. Hence, any known mechanical connection may secure the sleeve 58 to the flexible tubular 36 member and the tip 54 to the other end of the sleeve 58, thus securing the balloon 48 therebetween. It should be noted that any type of orifice as described above may be arranged within the extension 56 of the tubular member 36 of the cervical dilator 10 to allow for introduction of a warm saline solution or gas therein by the physician to inflate the balloon 48 to a predetermined diameter to create full cervical dilation. It should be noted that generally all of the parts described above are made of a sturdy plastic material, however any other ceramic, composite, metal, rubber or natural material may also be used for any of these parts described above. It also should be noted that any known mechanical securing technology may be used to secure the sleeve 58 to the tubular member 36 and the tip 54 to the sleeve 58 other than those described above.

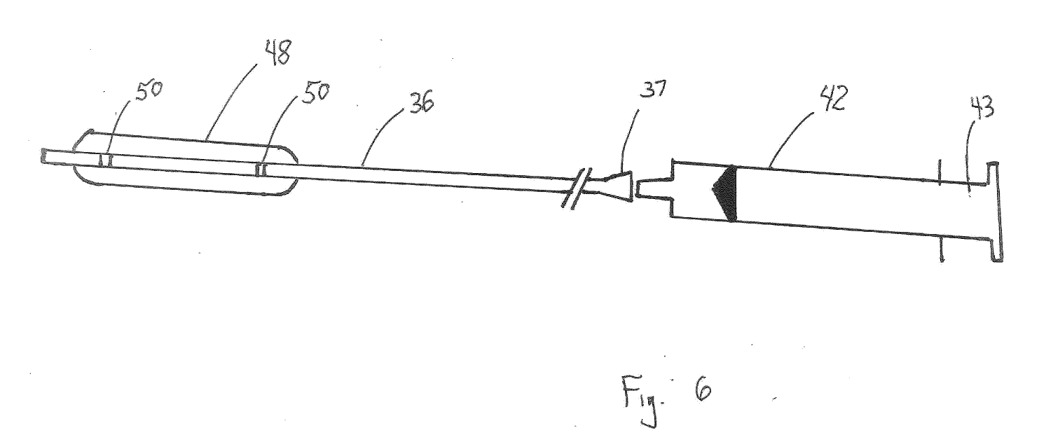

FIG. 6 shows an embodiment of the cervical dilator of the present invention that includes a syringe 42 having a plunger 43 therein, like the syringes 42 described above. It should be noted that in one contemplated embodiment the syringe 42 may have a possible volume of between two and twenty cubic centimeters. The syringe 42 may be used to introduce a substance, such as a liquid or air into a flexible tube member 36 and then into a balloon 48 arranged on an end thereof. The alternate embodiment flexible tube member 36 has a first end and a second end. The first end of the flexible tube member 36 may have a port 37 like tapered end on the first end thereof. The tapered end 37 generally may taper in an outward direction, such that it increases the diameter of the first end of the flexible tube member 36 at a predetermined rate. This port 37 on the first end of the flexible tube member 36 may be used for receiving the nipple or tip end of the syringe 42 therein. It should be noted that any known securing technique between the syringe 42 and the first end 37 of the flexible tube 36 may be used, such as but not limited to a locking/snap feature, a press fit connection, a threaded connection, etc. It should be noted that generally the flexible tube 36 may be the same as those described above and have a predetermined length. In one contemplated embodiment the length may be anywhere between fifteen centimeters and forty centimeters depending on the application. In one specific embodiment, it may have the length of approximately twenty centimeters with a forty millimeter portion arranged at the second end. The second end of the flexible tube member 36 may be sealed from the outside environment, either via a plug, an insert, an end cap or by a manufacturing technique that creates a solid wall, etc., at the second end of the flexible tube member 36. The flexible tube member 36 may be the same as that described above, i.e., made of a generally plastic material that is hypoallergenic and capable of being used in a surgery environment. As shown in FIG. 7 a balloon 48 may be arranged and secured over or near the second end of the flexible tube member 36. The balloon 48 may have any predetermined length and may be capable of expanding to any predetermined diameter. In one contemplated embodiment the balloon 48 may have any length of between twenty and sixty millimeters and a diameter between two and twenty millimeters. In one specific embodiment, the balloon 48 may have an approximate length of forty millimeters and may be capable of expanding to a fully expanded diameter of approximately fifteen millimeters. As described above, the balloon 48 may be secured on each end thereof to the outer surface of the flexible tube member 38 over or near the second end, i.e., a predetermined distance from the second end of the tube 36. The balloon 48 may be secured via any known mechanical or chemical securing methodology, such as but not limited to rubber bands, a pin and groove, a chemical fastening technique, such as glue, or welding, etc. Any known technique may be used to secure the balloon 48 on each end thereof to the outer surface of the flexible tube member 36. In the embodiment shown in FIG. 7 at least one orifice 50 is arranged through the surface of the flexible tube member 36 such that the orifice 50 is arranged within the inner cavity of the balloon 48 arranged thereover. in the embodiment shown a first and second orifice 50 may be arranged in the tube member 36 within the balloon 48. The first and second orifices 50 are arranged a predetermined distance from the second end and from each other through the surface of the flexible tube member 36. Hence, as described above and hereafter, after a liquid, such as a warm saline solution, is introduced via the syringe 42 into the tube 36 the warm saline solution may pass through the orifices 50 arranged near the second sealed end of the flexible tube member 36 and Inflate the balloon 48 to a predetermined diameter, thus dilating the cervix of the woman to allow for the physician to see through into the uterus and discover the source of any potential problems therein. When the physician has completed the dilation as described above, the physician may pull back on the plunger 43 thus removing the liquid from inside the balloon 48, via the orifices 50 in the flexible tube member 36, back into the syringe 42 thus deflating the balloon 48 and allowing for the physician to remove the cervical dilator 10 and insert any other instrument needed to perform the procedures on the uterus therein. It should be noted that in one contemplated embodiment the flexible tube 36 may have a diameter of approximately two millimeters, however any other diameter ranging from one half millimeter up to twenty millimeters may also be used.

Therefore, during the process of using the improved cervical dilator 10 of the present invention the physician may introduce the flexible tubular member 36 having an uninflated balloon 48 secured over the end thereof into the endometrial canal 30 of the patient via the cervix 14. The physician may measure the length of the uterus 28 via markings arranged on an outer surface of rise tubular member 36 of the cervical dilator 10 and may position an adjustable ring 52 to help secure the balloon 48 on one end in one contemplated embodiment thereof. In another contemplated embodiment thereof the physician may insert a multi piece end and balloon and will not have to use a ring or collar to secure the end of the balloon 48 to ensure an air tight seal for the balloon 48. The physician may be able to measure the length of the uterus via demarcations or markings on the outer surface of the flexible tubular member 36. Once the balloon 48 of the flexible tubular member 36 is inserted into the endometrial canal 30 of the patient, the physician via the syringe 42 or any other member capable of passing air or any liquid into the flexible tubular member 36 of the cervical dilator 10, depresses the plunger of the syringe 42 thus inserting in one contemplated embodiment a warm saline solution into the balloon 48 and inflating the balloon 48 at predetermined increments and at predetermined times to dilate the cervix 14 to the appropriate size in order for the physician to look through the cervical canal 14 to either perform a procedure or identify the source of bleeding from the uterus 28. Upon complete dilation of the cervix 14 to the diameter sufficient for the examination to occur, the physician may pull back on the plunger of the syringe 42 thus removing the air or liquid from inside the balloon 48 back into the syringe 48 thus allowing for removal of the cervical dilator 10 and insertion of other instruments to perform the procedure or examination of the now visible uterus 28. It should he noted that the use of the single insertion and balloon 48 to dilate the cervix 14 may reduce the risk of perforations, contaminations and also reduce the time of the procedure for the patient thus making the patient more comfortable and lessen the risk of damage to all surrounding organs and vessels of the patient. It should be noted that the balloon diameter may be capable of expanding anywhere from one to twenty millimeters depending on the patient and the procedure being performed.

The present invention has been described in an illustrative manner. It is to be understood that the terminology, which has been used, is intended to be in the nature of words of description rather than that of limitation.

Marry modifications and variations of the present invention are possible in light of the above teachings. Therefore, within the scope of the appended claims, the present invention may be practiced otherwise than as specifically described.

What is claimed is:

1. A cervical dilator, said dilator comprising:
a tube having a first end and a second end;
a syringe removably connected to said first end;
a balloon arranged over said tube at or near said second end; and
at least one orifice in said tube, said orifice is covered by said balloon.

2. The dilator of claim 1 wherein said first end having a tapered end, said taper increases a diameter of said first end.

3. The dilator of claim 1 wherein said tube is made of a plastic.

4. The dilator of claim 1 wherein said syringe is capable of delivering a warm saline solution into said tube.

5. The dilator of claim 1 further comprising additional orifices in said tube, said additional orifices are covered by said balloon.

6. The dilator of claim 5 wherein said one orifice is located near a first end of said balloon, said second orifice is located near a second end of said balloon.

7. The dilator of claim 1 wherein said second end of said tube is sealed closed.

8. The dilator of claim 1 wherein said balloon is secured on each end to an outer surface of said tube, said balloon is capable of being inflated via said syringe to a predetermined diameter.

9. The dilator of claim 1 wherein said tube is flexible.

10. The dilator of claim 1 further comprising a potential collar arranged over an outer surface of said tube and said balloon, said collar is movable with relation to said second end of said tube.

11. The dilator of claim 1 wherein said balloon is uninflated before use of the dilator.

12. The dilator of claim 1 wherein said tube having markings on an outer surface thereof to measure a length of a cervix.

13. A method of dilating a cervix of a patient with a single cervical dilator, said method comprising the steps of:
introducing a flexible tube having an uninflated balloon over an end thereof into the cervix;
connecting a syringe to said flexible tube;
passing a substance into said flexible tube via the syringe; and
inflating said balloon via said substance to a predetermined diameter, said inflated balloon dilates the cervix to a predetermined diameter.

14. The method of claim 13 further comprising the step of measuring a uterus via markings on an outer surface of said flexible tube.

15. The method of claim 13 wherein, said syringe having a plunger, said plunger is pressed inward to pass said substance at potentially predetermined increments and times into said balloon in order to dilate the cervix.

16. The method of claim 15 wherein said plunger is pulled back in order for said syringe to receive said substance therein, said balloon deflates by removal of said substance therein allowing for removal of the cervical dilator from the patient.

17. The method of claim 13 wherein said balloon is secured on each end thereof to an outer surface of said flexible tube.

18. The method of claim 13 wherein said balloon is arranged over at least one orifice arranged, through a surface of said flexible tube.

19. The method of claim 13 wherein said substance is air or liquid.

20. The method of claim 13 wherein said syringe is removably connected to an end of said flexible tube.
